(19) United States
(12) Patent Application Publication
(10) Pub. No.: US 2004/0226843 A1
(43) Pub. Date: Nov. 18, 2004

(54) Packing wrapper for individually packed absorbent articles

(75) Inventors: Sofia Hermansson, Vastra Frolunda (SE); Anders Stenberg, Onsala (SE); Ing-Britt Magnusson, Molnlycke (SE)

Correspondence Address: BURNS DOANE SWECKER & MATHIS L L P, POST OFFICE BOX 1404, ALEXANDRIA, VA, 22313-1404, US

(73) Assignee: SCA Hygiene Products AB

(21) Appl. No.: 10/785,011

(22) Filed: Feb. 25, 2004

Related U.S. Application Data
(60) Provisional application No. 60/450,355, filed on Feb. 28, 2003.

(51) Int. Cl. A61F 13/15

(52) U.S. Cl. 206/440

(57) ABSTRACT

A packing wrapper (17) for an individually packed absorbent article (1), such as a sanitary towel, a panty liner or an incontinence pad, intended to enclose the absorbent article (1), the packing wrapper (17) includes an opening flap (31), and the opening flap (31) having a free edge (33). The packing wrapper also includes an element (35; 40) for tactile detection of the position of the free edge (33), the element (40) for tactile detection having a longitudinal extent essentially parallel to the free edge (33) of the opening flap (31).

### CROSS-REFERENCE TO RELATED APPLICATIONS

The present application claims the benefit of U.S. Provisional Application No. 60/450,355, filed in the United States on Feb. 28, 2003, the entire contents of which are hereby incorporated herein by reference.

### BACKGROUND OF THE APPLICATION

1. Technical Field

The invention relates to a packing wrapper for an individually packed absorbent article, such as a sanitary towel, a panty liner or an incontinence pad, intended to enclose the absorbent article. The packing wrapper comprises an opening flap, the opening flap having a free edge.

2. Background Art

It is advantageous for several reasons to pack single absorbent articles of the kind referred to in the introduction in individual packing wrappers. In this way, small, handy packs are obtained, which can easily and conveniently be kept in a handbag or in a pocket and from which an article can be removed when necessary. Such individual packs ensure that the article is protected against soiling and crumpling until use and are therefore greatly appreciated by users. With suitable design of the packing wrappers, these can also serve as wrappers for used articles, which can thus be disposed of in a hygienic and aesthetically acceptable way.

One type of individually packed absorbent article is described in the document GB 2,153,779, where an absorbent article is placed in a centred position on a piece of material constituting the packing wrapper of the article, the packing wrapper extending outside the longitudinal and transverse edges of the absorbent article. The absorbent article is then folded together with the packing wrapper around two transverse fold lines arranged so that the article and the packing wrapper form three plies of essentially the same extent lying one on another. The parts of the packing wrapper which in this connection extend outside the two longitudinal edges of the absorbent article have, after folding, three plies of packing material arranged one on another. Finally, these three plies are connected by means of a conventional technique such as thermal welding, gluing, ultrasonic welding or the like. The connection is also designed so that it can be broken easily when the pack is to be opened.

One problem with individually packed absorbent articles of the kind described above arises when the pack is to be opened, as the user has to find where on the pack the opening is located.

The most common type of material used as packing wrappers for absorbent articles of the kind described is thin coloured plastic films, which are normally tightened relatively firmly around the absorbent article located inside the packing wrapper. The free edge of the opening flap then lies very closely against underlying material and is therefore extremely difficult to detect.

There are individual packs where the edge of the opening flap is provided with a different colour compared with the background colour, that is to say the basic colour of the packing wrapper, in order to facilitate detection of the free edge of the opening flap.

A large group of users of individually packed absorbent articles, especially of the incontinence pad type, is elderly, often weakly sighted users. For these users, the problem of locating the opening of the individual pack is even greater than it is for younger users with normal touch and sight. Consequently, solutions using colour-marking of the opening do not function satisfactorily for these users.

Also common are individually packed absorbent articles where the absorbent article is folded separately in a first folding operation along one or more fold lines at right angles to the longitudinal direction of the article. The folded absorbent article is then enclosed by a packing wrapper which is wider than the absorbent article, the packing wrapper then extending outside the edges of the absorbent article. Finally, the parts of the packing wrapper which extend outside the absorbent article are sealed so that a closed individually packed article is formed. The difficulties described above of detecting where the opening of the packing wrapper is located also arise for this type of individual pack, especially for weakly sighted people.

A third type of individually packed absorbent article is described in inter alia patent EP 0,865,264. In order to eliminate the use of the separate release-agent-treated protective layers which are usually used in order to cover the glued surface areas which are arranged on absorbent articles and are intended to fix the article to the briefs of the wearer during use, the inside of the packing wrapper is treated with a release agent. The glued area of the absorbent article is then placed directly against the release-agent-treated inside of the packing wrapper before folding and sealing of the absorbent article together with the packing wrapper are carried out. The packing wrapper described in EP 0,865,264 has a special tape tab arranged on the outside of the packing wrapper and intended to improve closing of the pack. Such tape tabs are arranged primarily in order to facilitate closing of a used absorbent article placed inside the packing wrapper before its disposal. The tape tabs also facilitate detection and opening of the packing wrapper because the position of the free edge of the opening flap can be detected via the tape tab by means of both sight and touch. This detection possibility is advantageous when a person with reduced ability to see is to open the packing wrapper.

A tape tab on the packing wrapper according to the description above nevertheless involves a number of disadvantages, the main one being the extra material cost the tape tab involves. Another major disadvantage is that the manufacturing rate is reduced drastically as separate tape tabs have to be applied to the packing wrapper, which also has a negative effect on the cost of the individually packed absorbent article.

A need therefore remains for an improved wrapper for an individually packed absorbent article where the opening on the wrapper can be detected by means of touch. A need also exists for a packing wrapper which is inexpensive to manufacture and can moreover be manufactured at high rates.

### SUMMARY

By virtue of the present invention, an article of the kind referred to in the introduction has been produced, which article reduces or eliminates the problems associated with previously known packing wrappers for individually packed absorbent articles.

A packing wrapper, made according to the invention, for an individually packed absorbent article of the kind referred to in the introduction is in this connection characterized mainly in that the means for tactile detection has a longitudinal extent essentially parallel to the free edge of the opening flap.

In this connection, the opening flap can comprise a surface area which comprises the means for tactile detection of the position of the free edge of the opening flap.

Alternatively, or in addition to a means for tactile detection arranged on the opening flap, a surface area which is not located on the opening flap can comprise the means for tactile detection of the position of the free edge of the opening flap. In this connection, the means for tactile detection is suitably arranged on the material of the packing wrapper adjacent to the free edge of the opening flap.

In order to make tactile detection possible along a large part of the free edge length of the opening flap, the means for tactile detection can consist of a continuous oblong surface area which is arranged parallel to and close to the free edge of the opening flap.

In an alternative embodiment, the means for tactile detection can consist of discrete surface areas which are arranged parallel to and close to the free edge of the opening flap.

A means for tactile detection which is particularly simple and inexpensive to produce consists of a surface area on the packing wrapper which comprises an embossed design.

The means for tactile detection can also consist of at least one surface area on the packing wrapper which is provided with an extra material piece.

The means for tactile detection can alternatively consist of a surface area on the packing wrapper which is coated with a material which has higher or lower friction than the packing wrapper.

As the packing wrapper is often made from a plastic film, it is also possible for the extra material piece to consist of a nonwoven or paper material applied to the packing wrapper, it then being easy to detect the free edge of the opening flap by touch when a comparison is made with the plastic film of the wrapper material.

The extra material piece can also consist of one or more thread-shaped materials which are also easy to detect by means of touch when they are arranged on a plastic film.

The extra material piece can have greater rigidity than the packing wrapper.

The means for tactile detection can also be produced by virtue of the free edge of the opening flap being folded one or more times.

In one embodiment, in order to make use of differences between the texture of the opening flap on its outside and its inside, the outermost part of the opening flap is folded so that that surface of the wrapper material which is mainly arranged inwards towards the absorbent article is exposed outwards at the free edge of the opening flap, the surface then constituting the means for tactile detection.

In another embodiment, the means for tactile detection extends over more than 30% of the length of the free edge of the opening flap. The means for tactile detection preferably extends over more than 50% of the length of the free edge of the opening flap.

### BRIEF DESCRIPTION OF FIGURES

The invention will be described in greater detail below with reference to the illustrative embodiments shown in accompanying figures, in which

FIG. 1 shows an incontinence pad positioned on a packing wrapper before folding together;

FIG. 2 shows the incontinence pad and the packing wrapper in FIG. 1, after a first folding-together;

FIG. 3 shows the incontinence pad and the packing wrapper in FIGS. 1 and 2 after a second, final folding-together;

FIG. 3b shows an alternative embodiment of the incontinence pad and the packing wrapper in FIGS. 1 and 2 after a second, final folding-together.

FIG. 4 shows an embodiment of the incontinence pad and the packing wrapper in FIGS. 1 and 2 after a second, final folding-together;

FIG. 5 shows a cross section along the line V-V in FIG. 4 of one embodiment, and

FIG. 6 shows a cross section along the line V-V in FIG. 4 of an alternative embodiment.

### DETAILED DESCRIPTION OF THE PREFERRED EMBODIMENTS

The invention relates to a packing wrapper for an individually packed absorbent article of the sanitary towel, panty liner, incontinence pad or baby diaper type. In the following description of various embodiments of the invention, it is to be understood that the invention is not limited to the absorbent articles shown in the figures, but that the articles indicated above are freely interchangeable.

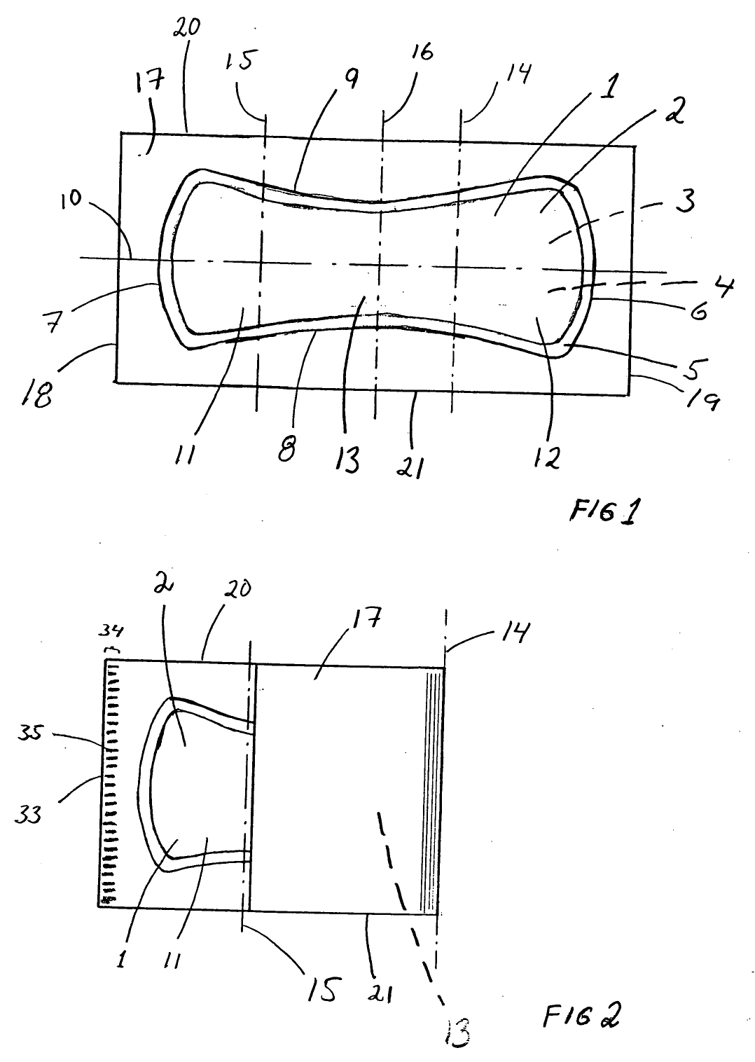

The incontinence pad 1 shown in FIG. 1, intended for lighter forms of incontinence, or mild incontinence, has a conventional construction and comprises a first, liquid-permeable covering layer 2, arranged on that side of the incontinence pad 1 which is intended to face the wearer during use, a second, liquid-impermeable covering layer 3, arranged on that side of the incontinence pad 1 which is intended to face away from the wearer during use, and an absorbent body 4 enclosed between the two covering layers 2, 3.

The absorbent body 4 can be constructed from one or more layers of cellulose fluff pulp. In this connection, cellulose fluff pulp can be mixed with fibres or particles of a highly absorbent polymer material of the kind which chemically binds great quantities of liquid during absorption while forming a liquid-containing gel. The absorbent body 4 can also comprise highly absorbent polymer material arranged in one or more layers inside the absorbent body or adjacent to the surface or surfaces of the absorbent body. The absorbent body 4 can also include components of a non-absorbent nature in order to improve the properties of the absorbent body 4. Examples of such components are bonding fibres, shape-stabilizing components, strengthening fibres or the like. Various types of layer and material provided for improved spreading of liquid can also form part of the absorbent body 4. The absorbent body 4 can of course comprise other types of absorption material, such as absorbent nonwoven materials, absorbent foams, textile materials, peat, or mixtures of different kinds of absorption material.

Special layers for rapidly receiving large quantities of liquid and temporarily storing this liquid in order then to discharge the temporarily stored liquid to other parts of the absorbent body 4 can also form part of the incontinence pad 1. Such receiving layers are normally arranged between the liquid-permeable covering layer 2 and the absorbent body 4 of the incontinence pad 1.

The two covering layers 2, 3 project past the edges of the absorbent body 4, and the projecting portions 5 of the covering layers are interconnected around the periphery of the absorbent body 4, for example by means of gluing, sewing, or by welding using heat or ultrasound.

The material in the liquid-permeable covering layer 2 can be of any suitable kind. Examples of common liquid-permeable covering materials are various types of bonded nonwoven materials, perforated plastic films, net and also open-cell or perforated foam materials. Liquid-permeable covering materials which consist of continuous thin fibres which extend mainly in the longitudinal direction or transverse direction of the absorbent article are also found. Laminates consisting of two or more of the abovementioned possible covering materials are also common, as are coverings consisting of different materials in different parts of the surface.

An incontinence pad which comprises absorbent bodies 4 with particularly great strength and wear-resistance can even function without any extra liquid-permeable covering layer being required on that side of the incontinence pad which faces the wearer during use.

The liquid-impermeable covering layer 3 suitably consists of a thin plastic film, or of a nonwoven material which has been made liquid-impermeable by coating or treatment with a material which resists liquid penetration. Other types of liquid-blocking material can of course also be used, such as, for example, plastic foam with closed cells, various liquid-blocking laminates etc. In order that the incontinence pad 1 will feel airy and pleasant to wear, it is suitable for the liquid-impermeable covering layer 3 to have a certain permeability for air and water vapour. The liquid-impermeable covering layer 3 can also be integrated in the absorbent body 4 and consist of, for example, a skin-like surface on an absorbent foam body.

The incontinence pad 1 is elongate and has an hourglass shape and is delimited in the plane by two transverse end edges 6, 7 and longitudinal side edges 8, 9 extending between these. The incontinence pad can also be divided along its longitudinal symmetry line 10 into a first and a second end portion 11, 12 and a central portion 13 located between the end portions 11, 12. The various portions 11-13 are not clearly defined in relation to one another but, roughly speaking, they can each be assumed to occupy approximately a third of the length of the incontinence pad 1. The central portion 13, also called the crotch portion 13, is usually considered to constitute that part of the incontinence pad 1 within which what is known as the wetting area is located.

The wetting area is that portion on the incontinence pad 1 which is intended first to receive discharged urine or other bodily fluid. The size of the end portions 11, 12 can vary slightly, but in most cases that end portion facing backwards on the wearer during use is considered to be slightly longer than the end portion facing forwards. As the end portions 11, 12 on the incontinence pad 1 shown in FIG. 1 are entirely symmetrical, the incontinence pad 1 can be worn with either of the two end portions 11, 12 facing forwards during use. The size and the position of the various portions 11-13 of the incontinence pad along the longitudinal symmetry line 10 is then not determined until use.

In FIGS. 1-3, two transverse fold lines 14, 15 have been drawn in, one on either side of the transverse centre line 16 of the incontinence pad 1. The end portions 11, 12 and central portion 13 of the incontinence pad 1 essentially coincide with the division along the longitudinal centre line 10 of the incontinence pad 1 which is defined by the fold lines 14, 15.

Arranged on the outside of the liquid-impermeable covering layer 3 is a fastening arrangement (not visible in the figure) in the form of at least one area of pressure-sensitive self-adhesive glue. The fastening arrangement is attached detachably to one surface of a rectangular packing wrapper 17. Common fixing glue patterns consist of one or more longitudinal adhesive strands, or two or more transverse glue strands. Other patterns can also be used, however, as can full coating of the liquid-impermeable covering layer 3 with glue.

Like the incontinence pad, the packing wrapper 17 has two transverse end edges 18, 19 and two longitudinal side edges 20, 21; it also has a greater extent than the incontinence pad 1, as a result of which it projects outside the edges 6-9 of the incontinence pad 1 around its entire periphery.

The packing wrapper 17 is formed from a sheet or, for example, paper, plastic or another suitable flexible material, which has been release-agent-treated on the surface facing the incontinence pad 1 in order to bring about the desired detachable fastening of the fastening arrangement of the incontinence pad 1. Such release-agent treatment usually consists of silicone coating. The release-agent treatment can be applied over the entire surface of the packing wrapper 17 facing the incontinence pad 1 or be applied selectively only within those areas which lie against the fastening arrangement of the incontinence pad 1.

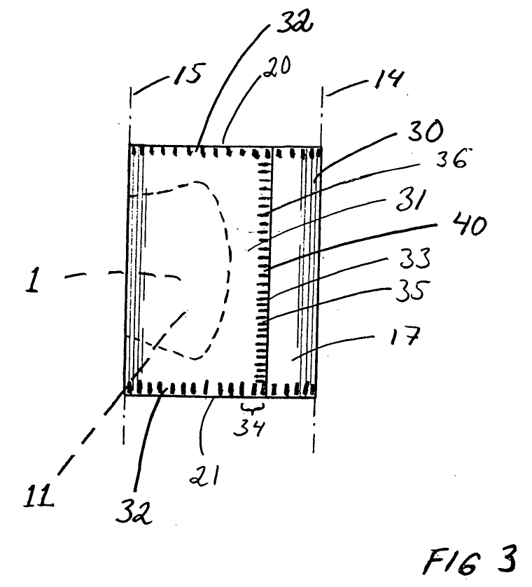

FIG. 2 shows how the incontinence pad 1 and the packing wrapper 17 appear after a first folding-together. The second end portion 12 of the incontinence pad 1 has, together with the packing wrapper 17, been folded in over the central portion 13 of the incontinence pad 1 along the first transverse fold line 14. Folding has been effected in the direction in over the liquid-permeable covering layer 2 of the incontinence pad 1, so that the liquid-permeable covering layer 2 in the second end portion 12 of the incontinence pad 1 lies against the liquid-permeable covering layer 2 in the central portion 13 of the incontinence pad 1 and the packing wrapper 17 faces outwards.

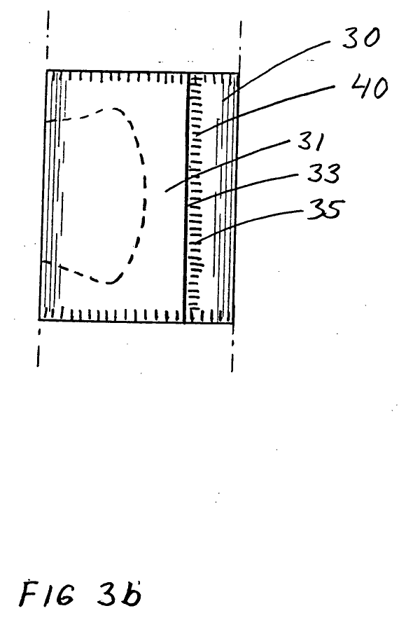

In FIG. 3, the incontinence pad 1 and the packing wrapper 17 are folded together again, along the second transverse fold line 15. The first end portion 11 of the incontinence pad 1 is then, together with the packing wrapper 17, folded in over the second end portion 12 folded in first, to form a handy pack format.

The folded-together pack 30 is sealed by virtue of the folded-together side edges 20, 21 of the packing wrapper 17 being welded together using heat or ultrasound. The weld joints 32 are suitably made in such a way that the pack 30 can be opened without the packing wrapper 17 being torn apart in an uncontrolled manner. This can be achieved by virtue of, for example, one surface included in the weld joint 32 being release-agent-treated, or by virtue of the joint 32 deforming the material in the packing wrapper 17 in such a way that the joint 32 serves as a tear indication for opening the pack 30.

When the pack 30 shown in FIG. 3 is to be opened, the folding-together operations performed during formation of the pack 30 are repeated in reverse order. That end portion 11 of the incontinence pad folded in last is therefore folded out together with the packing wrapper 17. In this connection, the closure of the edges 20, 21 of the pack 30 is broken by virtue of the packing material being torn apart along the weld joints 32, or by virtue of the weld joints 32 being opened. When that end portion 12 of the incontinence pad 1 folded in first and the packing wrapper 17 are then folded out, the remaining edge closure 32 of the pack 30 is broken. The incontinence pad 1 now has the same configuration as shown in FIG. 1, that is to say lying flat and attached detachably to the packing wrapper 17 by the fastening means arranged on the liquid-impermeable covering layer.

A pack 30 according to the invention is characterized mainly in that it comprises means 40 for tactile detection of the position of the opening flap 31.

The individually packed incontinence pad 1 shown in FIG. 3 is characterized in that the opening flap 31 of the pack 30 can be detected by means of touch. In this connection, the opening flap 31 comprises a free edge 33 and an edge area 34 adjoining the free edge 33. The edge area 34 extends along the entire free edge 33 and comprises an embossed design 35 so that the opening flap 31 of the pack 30 can be detected easily by means of the sense of touch in, for example, the fingers.

The embossed design 35 has a width which is the same as the width of the edge area 34 and extends along the entire free edge 33 of the opening flap, but can also consist of a number of shorter discrete embossed designs positioned along the free edge 33 of the opening flap 31.

In alternative embodiments, the embossed design 35 can extend along only a part of the free edge 33 of the opening flap 31. In this connection, the embossed design suitably extends over more than 30% of the length of the free edge 33, preferably more than 50% of the length.

The width of the edge area is suitably 0.1-4 cm, preferably 0.3-1.5 cm.

The embossed design 35 is brought about by virtue of the packing wrapper 17 having passed through an embossing unit before the last folding is performed around the second transverse fold line 15. Embossing is effected by virtue of the material of the packing wrapper 17 passing through a unit consisting of a male part with projecting embossing bodies and female part with sunken embossing recesses arranged synchronously in relation to one another. When that part of the material of the packing wrapper 17 to be embossed, that is to say the edge area 34, passes between the male part and the female part of the embossing unit, the material of the packing wrapper 17 is deformed plastically, permanently deformed protuberances being obtained in the material. Alternative ways of bringing about embossing can also be envisaged.

In the illustrative embodiment, the pattern of the embossed design 35 consists of straight embossed lines 36 which extend at right angles to the free edge 33 of the opening flap and with an extent within the edge area 34.

The pattern of the embossed design can vary within wide limits and can consist of, for example, lines parallel to the free edge of the opening flap, small circles in the edge area, figures, symbols or the like.

Embossing the edge area 34 affords the great advantage that no costly extra material has to be added, because it is the already present packing wrapper 17 which is modified so that detection is made possible using the sense of touch in the fingers. Another advantage of embossing is that the materials most frequently used for packs 30 for absorbent articles 1, such as thin plastic films or paper, can be embossed at high machine rates, which means that embossing does not involve more expensive manufacture either.

The pack material must, at least in the area to be embossed, comprise an embossable material. Suitable embossable materials are various types of plastic film, paper or the like.

As an alternative to an embossed edge area 34, it is possible to envisage an edge area 34 which is perforated or the like and can in this way be detected easily by means of the sense of touch in the fingers. As the opening flap 31 overlaps the basic material of the pack 30, such holes will not constitute openings into the pack but only openings in towards underlying basic material of the pack 30.

The folded-together pack 30 shown in FIG. 3b is a pack 30 with an alternatively positioned means 40 for tactile detection. The means 40 consists of an embossed surface area 35 in this embodiment as well. The embossed design 35 is arranged next to and along the free edge 33 of the opening flap 31 on that side of the free edge 33 which does not belong to the opening flap 31. It is also conceivable to arrange embossed designs 35 next to the free edge 33 both on the opening flap 33 and on that side of the free edge 33 which does not belong to the opening flap 31, the free edge 33 of the opening flap 31 then being located between the two embossed surface areas 35.

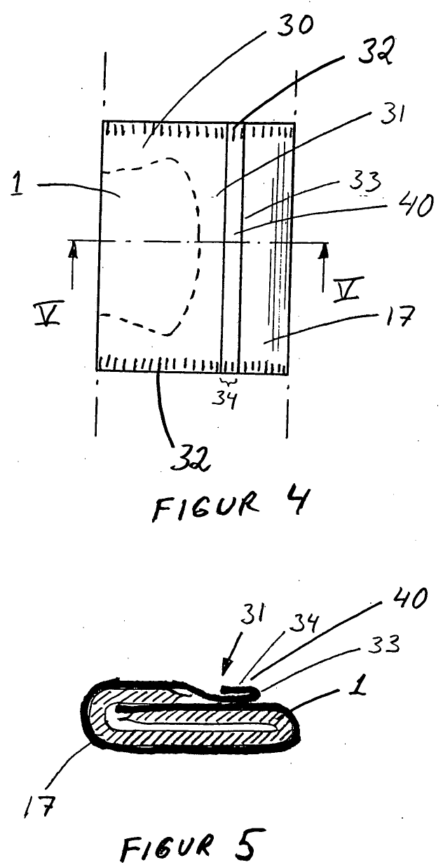

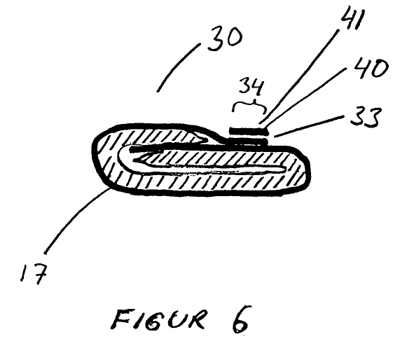

FIGS. 4 and 5 show another alternative embodiment of an individually packed incontinence pad 1 in accordance with the invention. FIG. 5 shows a cross-sectional view of the individually packed incontinence pad 1 in its folded-together pack 30 at the line V-V in FIG. 4.

In this embodiment, the opening flap 31 of the folded-together pack 30 has been double-folded in the edge area 34. The opening location of the individual pack 30 can then be located using the sense of touch in the fingers as it is easy to feel that the opening flap 31 is double-folded and thus thicker than surrounding areas.

It is also possible to fold the opening flap 31 several times in the edge area 34 in order to create an even greater difference in thickness compared with surrounding areas. The opening flap 31 can also be folded or rolled in alternative ways in the edge area 34 in order to produce a free edge 33 which can be detected by touch.

In order to fix the folded material plies in the edge area 34 in their position, the fixing brought about by the weld joints 32 is sufficient, but it is also possible to envisage strengthening the fixing by applying glue or the like between the folded material plies.

Packing wrappers 17 of the type which is release-agent-treated on the surface facing towards the absorbent article usually have considerably lower friction on the release-agent-treated side than on the opposite side. It is therefore suitable to arrange the folding in the edge area 34 so that the release-agent-treated surface is exposed outwards on the folded individual pack 30 and in this connection, in addition to the difference in thickness produced, also to obtain a difference in friction between the edge area 34 and surrounding packing wrapper 17.

Many other types of packing material, such as non-release-agent-treated plastic, paper or the like, also normally have different friction on their opposite surfaces, for which reason the differences in friction described above can also be made use of for packing wrapper material 17 which is not release-agent-treated, although the difference in friction between the two sides of the packing wrapper material 17 is apt to be smaller than for packing materials which are release-agent-treated on one of their sides.

FIG. 6 shows a cross-sectional view along the line V-V in FIG. 4 of another embodiment of the invention. In accordance with this embodiment, the edge area 34 of the folded-together pack 30 has been provided with an extra material strip 41 extending along the entire free edge 33 of the individual pack.

The material strip 41 is connected to the packing wrapper material 17 by means of gluing, thermal welding, ultrasonic welding or the like on that side of the packing wrapper 17 which faces away from the absorbent product inside the pack 30.

The material strip 41 consists of a material which has different properties with regard to touch compared with the surrounding wrapper material 17. Suitable materials are plastic strips with different friction properties, nonwoven strips, paper strips or the like.

The material strip 41 can of course also have another type of different texture in relation to the wrapper material 17; it can be, for example, an embossed material strip 41, a material strip 41 which is considerably more rigid than the wrapper material 17, a material strip 41 which is thicker than the wrapper material 17, or different in another way.

The material strip 41 can also consist of a thread-shaped material which can be detected easily by means of the sense of touch in the fingers. It is possible, for example, to envisage one or more threads arranged in the edge area 34 parallel to the free edge 33 of the opening and connected to the wrapper material 17. The threads can consist of textile threads, rubber threads or the like, which differ from the texture of the wrapper material 17 with regard to touch.

Instead of a material strip 41, it is conceivable to coat the edge area 34 with a material so that it has different properties, with regard to touch, compared with the wrapper material 17. The coating in the edge. area 34 can consist of, for example, rubber coating having elevated friction, solid particles which have been fixed in a glue film so that the surface of the edge area 34 feels rough, or the like.

The invention also comprises all conceivable combinations of the illustrative embodiments described.

Furthermore, the invention is not limited to the illustrative embodiments referred to above but is of course applicable for other embodiments within the scope of the following patent claims.

Although only preferred embodiments are specifically illustrated and described herein, it will be appreciated that many modifications and variations of the present invention are possible in light of the above teachings and within the purview of the appended claims without departing from the spirit and intended scope of the invention.

1. A packing wrapper for an individually packed absorbent article, the packing wrapper intended to enclose the absorbent article, the packing wrapper comprising an opening flap, and the opening flap having a free edge, wherein the packing wrapper comprises means for tactile detection of the position of the free edge, the means for tactile detection having a longitudinal extent essentially parallel to the free edge of the opening flap.

2. The packing wrapper according to claim 1, wherein at least one surface area on the opening flap comprises the means for tactile detection of the position of the free edge.

3. The packing wrapper according to claim 1, wherein at least one surface area close to the opening flap comprises the means for tactile detection of the position of the free edge.

4. The packing wrapper according to claim 1, wherein the means for tactile detection is applied to a continuous, oblong surface area arranged parallel to and close to the free edge of the opening flap.

5. The packing wrapper according to claim 1, wherein the means for tactile detection is applied to at least two discrete surface areas arranged parallel to and close to the free edge of the opening flap.

6. The packing wrapper according to claim 1, wherein the means for tactile detection consists of at least one embossed surface area.

7. The packing wrapper according to claim 1, wherein the means for tactile detection consists of at least one extra material piece.

8. The packing wrapper according to claim 1, wherein the means for tactile detection consists of at least one surface area on the packing wrapper which is coated with a material which has higher or lower friction than the packing wrapper.

9. The packing wrapper according to claim 7, wherein the extra material piece consists of a nonwoven material.

10. The packing wrapper according to claim 7, wherein the extra material piece consists of a paper material.

11. The packing wrapper according to claim 7, wherein the extra material piece consists of at least one thread-shaped material.

12. The packing wrapper according to claim 7, wherein the extra material piece has greater rigidity than the packing wrapper.

13. The packing wrapper according to claim 1, wherein the means for tactile detection is produced by virtue of the opening flap being at least double-folded at the free edge.

14. The packing wrapper according to claim 13, wherein the outermost part of the opening flap is folded, a surface of the opening flap which before folding was arranged towards the absorbent article being exposed outwards, this surface then constituting the means for tactile detection.

15. The packing wrapper according to claim 1, wherein the means for tactile detection extends over more than 30% of the length of the free edge of the opening flap.

16. The packing wrapper according to claim 1, wherein the means for tactile detection extends over more than 50% of the length of the free edge of the opening flap.

17. The packing wrapper according to claim 1, wherein the absorbent article is a sanitary towel, a panty liner, or an incontinence pad.
